(19) United States
(12) Patent Application Publication
(10) Pub. No.: US 2012/0330229 A1
(43) Pub. Date: Dec. 27, 2012

(54) SYRINGE-LIKE MIXING DEVICE HAVING A DISTALLY OPERABLE MIXING ELEMENT

(75) Inventor: Andy Greter, Baar (CH)

(73) Assignee: MEDMIX SYSTEMS AG, Rotkreuz (CH)

(21) Appl. No.: 13/580,227

(22) PCT Filed: Jan. 21, 2011
(86) PCT No.: PCT/CH2011/000006

§ 371 Date: Aug. 21, 2012

(30) Foreign Application Priority Data
Feb. 22, 2010 (CH) 219/10

(51) Int. Cl. A61M 5/31

(52) U.S. Cl. 604/82

(57) ABSTRACT

The invention relates to a syringe-like mixing device, comprising a housing, in which a mixing chamber is formed, a piston, which can be slid in the mixing chamber from the proximal side, and a mixing element, which can be operated from the distal side. In order to ensure simple operation, the mixing device has a sealing cap, which seals the distal open end of the mixing chamber and which has a passage opening, through which an operating rod for the mixing element is guided. The sealing cap can be completely removed from the housing together with the operating rod and the mixing element. In order to discharge the mixing product, a second sealing cap can be placed onto the housing. Accessory parts can be connected to said second sealing cap. The mixing product can then be discharged as with a conventional injection syringe.

### TECHNICAL FIELD

The present invention relates to a syringe-like mixing device, comprising a housing that delimits an essentially cylindrical mixing chamber, and a piston which is slidably arranged in the mixing chamber, wherein a mixing element is inserted into the mixing chamber from the distal end.

### PRIOR ART

In various technical fields, among them in various medical applications, it is necessary to mix two or more components immediately prior to their application. One important example relates to the production of a bone cement from a powdery component and a liquid monomer. The two components are stored apart from each other. The components are mixed only immediately prior to application, as a result of which a curing process or gelling process is initiated. Therefore the product resulting from mixing needs to be discharged promptly after the mixing process. Other examples include the production of a medical adhesive or of a medicament from two or more components.

From the prior art for this purpose mixing devices are known in which the components to be mixed are placed into a cylindrical mixing chamber and in this mixing chamber are mixed by means of a mixing element, wherein in these mixing devices the mixed product is subsequently discharged from the mixing chamber, by means of a piston, through a distal dispensing opening. In particular, mixing devices are known that are of a syringe-like design. In such mixing devices the mixing chamber is formed in a housing that can be held between two fingers of a hand, and the piston is connected to a piston rod that at its free end comprises a thumb rest for the thumb of the same hand. As a result of this the mixed product can be discharged after the mixing process in the same way as is the case with a commercially available injection syringe. Such mixing devices are particularly suitable for mixing and discharging relatively small quantities.

Such a syringe-like mixing device is, for example, disclosed in WO 2009/105905. In this device the mixing element is operated from the same (proximal) side as the piston. In order to be able to operate the mixing element independently of the piston, an operating rod for the mixing element is guided through the piston. A piston rod is hingeably connected to the operating rod. In order to advance the piston, the piston rod is aligned so as to be parallel to the operating rod, wherein said piston rod in some regions radially encompasses the operating rod and engages the piston. In contrast to the above, in order to mix the product to be mixed, the piston rod is aligned across the operating rod. In this orientation the piston rod is held by the user and is used as a hand grip for moving the operating rod to and fro. However, an operating rod guided through the piston is not suitable for very small mixing chambers, because the diameter of the operating rod becomes too large relative to the piston diameter, and consequently such a device is sub-optimal from the point of view of ergonomics.

From prior art syringe-like mixing devices are also known in which the operating rod is inserted into the mixing chamber through the distal end, i.e. the end facing away from the piston. Such a device is, for example, disclosed in WO 2009/048366. In this mixing device the operating rod is hollow and is used for supplying at least one component to the mixing chamber, in which mixing chamber some other component is already present. In order to discharge the finished product, the operating rod is separated from the mixing element while the mixing element remains in the mixing chamber. Subsequently a hollow needle for discharging the mixture is arranged on the distal end of the mixing device. This device is associated with a disadvantage in that further components can only be inserted into the mixing chamber through the hollow operating rod, which, due to its construction, has a relatively small interior diameter. Consequently, such a mixing device is not suitable for subsequent insertion, for example, of highly-viscous or powdery components into the mixing chamber. Moreover, handling of the mixing device is relatively cumbersome. Furthermore, the need to design the interior of the operating rod for the mixing element so that it is hollow adversely affects the stability of the operating rod.

For handling larger quantities of a product to be mixed, mixing devices are known from prior art, in which mixing devices the mixed product is discharged with the use of a pistol dispenser or similar means. Examples of such devices are disclosed, for example, in U.S. Pat. No. 5,842,785 or US 2005/0128867. Due to their size and complexity such devices are, however, not suitable for the production and discharge of small quantities of a mixed product.

### SUMMARY OF THE INVENTION

It is thus an object of the present invention to provide a mixing device for mixing a product to be mixed, which mixing device is of a simple design, is suitable for the production of small quantities of a mixed product, from which mixing device the mixed product can easily be discharged in the manner of an injection device, and which mixing device can be filled in a simple manner with the components to be mixed.

This object is met by a mixing device with the features of claim 1. Further embodiments of the mixing device are laid down in the dependent claims.

Thus a mixing device for mixing a product to be mixed, in particular at least two components, is proposed, which mixing device comprises the following characteristics:
a housing that delimits an essentially cylindrical mixing chamber, wherein the mixing chamber defines a longitudinal direction and comprises a distal open end and a proximal open end, and wherein the housing is dimensioned in such a manner that it can be held between two fingers of a first hand of a user;
a piston arranged in the mixing chamber so as to be slidable along the longitudinal direction, which piston sealingly delimits the mixing chamber in the direction of the proximal end;
a piston rod, connected to the piston, which piston rod protrudes from the proximal end of the housing and which piston rod comprises a thumb rest, wherein the thumb rest is arranged in such a manner that it makes it possible for the user to advance the piston rod with the thumb of the first hand in the distal direction when the user holds the housing between two fingers of the first hand;
a mixing element that can be slid in the mixing chamber along the longitudinal direction;
an operating rod that is distally connected to the mixing element, which operating rod protrudes from the distal open end of the mixing chamber; and
a first closure cap that at the distal open end of the mixing chamber detachably engages the housing, which closure cap closes the distal open end of the mixing chamber and comprises a passage opening through which the operating rod is guided.

In this arrangement the mixing device is designed in such a manner that the first closure cap together with the operating rod and the mixing element can be completely removed from the housing.

In this manner on the one hand the components to be mixed can in a simple manner be placed in the mixing chamber, in particular also in those instances when one of the components is a highly-viscous fluid, a powdery substance or a plug-shaped solid substance. However, the mixing chamber can also already have been pre-filled under hygienic conditions with at least one component. On the other hand, the mixing element can in a very simple manner be removed from the mixing chamber so that the product can be discharged without the discharge process being impeded by the presence of the mixing element and the operating rod. At the same time such a device makes it possible to provide the operating rod with a hand grip, which ensures simple and ergonomic handling during mixing. The operating rod can readily be dimensioned in such a manner that the necessary stability of the operating rod is ensured at all times. In that in this arrangement the mixing device is otherwise designed in the manner of a normal injection syringe, the mixing device in addition offers extremely simple handling for the purpose of discharging the mixed product. Overall, sterile handling of the components during filling, mixing and discharging is ensured.

In order to ensure reliable guidance of the operating rod, the first closure cap can comprise a guide element whose length corresponds to at least a third of the internal diameter of the circular cylindrical mixing chamber, with said guide element preferably extending into the mixing chamber. In order to achieve good stability against tilting, the guide element preferably rests on at least three positions, preferably circumferentially, on the inside against a defining wall of the mixing chamber. The operating rod is guided in the guide element (which can also be referred to as a guide bush or guide sleeve). To this effect the guide element comprises a passage opening, in particular a through-hole, through which the operating rod is guided. Preferably, the passage opening directly serves to slidingly guide the operating rod and is long enough to prevent tilting of the operating rod relative to the guide element. Overall, in this manner particularly good stability of guidance of the operating rod is ensured.

Preferably, the first closure cap comprises a wiping lip that circumferentially rests against the operating rod in order to prevent the product to be mixed from leaking out during the mixing process. The wiping lip is preferably arranged on the guide element, in particular in a proximal end region of the guide element.

A proximal end region of the first closure cap, in particular of the guide element, can comprise an indentation that tapers off in the distal direction. This is advantageous in particular in those cases where the mixing element also tapers off in the distal direction. In this case the indentation receives the distal end of the mixing element when the mixing element is in a distal end position. In this manner it is ensured that the components received in the mixing chamber are efficiently mixed even in a distal region of the mixing chamber, which region directly adjoins the closure cap. If there is a wiping lip it is preferably formed in a region of the guide element, which region distally adjoins the indentation.

Correspondingly, the form of a distal end region of the piston can be designed so as to be complementary to the mixing element and, in particular, can comprise an end region that tapers off in the distal direction when the mixing element forms a receiving device for the end region of the piston, which receiving device tapers off in the distal direction. In this manner efficient mixing of the product to be mixed in a proximal end region of the mixing chamber, which end region directly adjoins the piston, is also ensured.

In one possible concrete embodiment the mixing element comprises at least one, preferably at least three, arms that are inclined in the proximal direction and that radially extend outwards from the central axis of the mixing chamber. The arms can be perceived as part of a rotational surface, in particular a surface of a cone, tapering toward the distal direction. In the region of the central axis of the mixing chamber the arms are connected to the operating rod. At their free ends the arms can comprise an opened-out shape and/or can comprise further structures; for example, at their free ends the arms can be of a T-shaped design. Irrespective of the concrete design of the mixing element, said mixing element preferably at least in some regions rests against the defining wall of the mixing chamber in order to ensure efficient mixing of the product to be mixed even in the region of the defining wall.

Preferably, the mixing element can not only be moved to and fro along the longitudinal direction within the mixing chamber, but it can also be rotated about the longitudinal direction. To this effect it is, preferably, moved manually. For this purpose the mixing device preferably comprises a hand grip that is connected to the operating rod at the latter's distal end that faces away from the mixing element. This hand grip is designed for the operating rod to be manually moved, by the second hand of the user, to and fro along the longitudinal direction, and to be rotated on the longitudinal direction, while the user holds the housing in the first hand. The hand grip can have been specially adapted to the anatomy of the human hand in order to ensure good ergonomics. Thus the hand grip can, for example, open out in a wedge-shaped manner in the distal direction, and can comprise an asymmetrical shape in relation to rotation on the longitudinal direction; in particular it can be flattened perpendicularly to the longitudinal direction.

Since the operating rod together with the closure cap and the mixing element is completely removable from the housing it is not necessary for the operating rod to be detachable from the mixing element. In order to be able to design and produce the mixing device in a particularly simple manner it is, in particular, preferred if the operating rod is non-detachably connected to the mixing element, in particular made in a single piece with the mixing element.

In order to affix the closure cap to the housing in a simple manner, an external thread can be formed on the housing in the region of the distal end of the mixing chamber. The closure cap then comprises a jacket region with an internal thread that engages the external thread, wherein the jacket region preferably encompasses the distal end of the housing. As an alternative, other solutions are also imaginable. For example, in particular, a bayonet guide in the form of a bayonet groove can be provided on the housing in the region of the distal end of the mixing chamber, and the closure cap can comprise a bayonet stud that engages the bayonet guide. Conversely, it is also possible for a bayonet stud to be provided on the housing, and for a bayonet guide in the form of a groove to be provided in the jacket region of the closure cap.

In order to be able to easily remove the mixing element from the mixing chamber even if said mixing element rests from the inside against the defining wall of the mixing chamber, the distal end of the mixing chamber is preferably designed in such a manner that it does not taper off in the distal direction relative to the rest of the mixing chamber. In other words, the distal end of the mixing chamber preferably forms an access opening to the mixing chamber, with the diameter of said access opening being at least as large as the diameter of the mixing chamber in a cylindrical region that is situated so as to be proximally spaced apart from the distal end, in which cylindrical region the mixing element is slidable.

Advantageously, the mixing device described above can be combined with further parts to form a mixing and discharge system. In particular, the mixing device can be combined with a second closure cap, which, after removal of the first closure cap, in the region of the distal open end of the mixing chamber can be made to detachably engage the housing. In this arrangement the second closure cap essentially closes the distal open end of the mixing chamber, and comprises a discharge opening through which a mixed product present in the mixing chamber can be discharged in the distal direction with the application of pressure on the piston rod.

Thus, normally, in order to discharge the mixed product the first closure cap together with the mixing element and the operating rod is removed from the housing, and the second closure cap is put in place. The mixed product can then be discharged through the discharge opening formed in the second closure cap.

The second closure cap can, in particular, be designed to make it possible for accessory parts to be connected to this cap. To this effect the second closure cap can comprise a connecting region for such an accessory part, which connecting region encloses the discharge opening. This connecting region preferably comprises a jacket surface that conically extends in the distal direction in order to connect the accessory part by way of a conventional conical surface connection. In particular, the connecting region is preferably designed as a male Luer cone, and to this effect comprises an inclination of approx. 6% relative to the longitudinal direction, according to the standards DIN EN 1707:1996 and DIN EN 20594-1:1993.

In order to secure the accessory part on the connecting region, the second closure cap can comprise a securing sleeve or union nut with an internal thread that points towards the connecting region, which securing sleeve or union nut radially encompasses the connecting region and is arranged, spaced apart from the connecting region, so as to be non-rotational or rotational. The connecting region and the securing sleeve can together be designed, in particular, as a Luer lock according to the above-mentioned standards.

In addition, the mixing system and discharge system can comprise a closure part that can be detachably attached to the connecting region of the second closure cap in order to close the discharge opening. This closure part can, in particular, comprise a sleeve with a female Luer cone, which sleeve can comprise an external thread that is complementary to the internal thread of the securing sleeve.

The mixing system and discharge system can, furthermore, comprise one or several accessory parts. The accessory parts can be detachably connectable to the discharge opening of the second closure cap, e.g. can be slipped onto the connecting region, or they can be non-detachably connected with the discharge opening and together with the second closure cap can form a unit. However, it is also possible for accessories to be directly integrated in the first closure cap. Possible accessory parts include, in particular: a discharge nozzle, a spraying device, a hollow needle or a mixing tube in which a further mixing element, e.g. a static mixing spiral or a dynamic mixing element, can be provided. Naturally, the above listing is not exhaustive. Furthermore, it is possible to remove the mixing unit after completion of the mixing procedure, and to discharge the mixture directly and without any accessories in place.

### BRIEF DESCRIPTION OF THE DRAWINGS

Below, preferred embodiments of the invention are described with reference to the drawings, which are provided only for elucidation and are not to be interpreted as being limiting. The following are shown in the drawings:

FIG. 1 a lateral view of a mixing device according to the invention;

FIG. 2 a perspective section view of the mixing device of FIG. 1;

FIG. 3 an enlarged section from FIG. 2;

FIG. 4 a perspective disaggregated view of the mixing device of FIG. 1;

FIG. 5 a lateral view of the housing and of the piston unit of FIG. 1, without a mixing unit;

FIG. 6 a lateral view of the housing and of the piston unit with a second closure cap;

FIG. 7 a lateral view of the device of FIG. 6 with an additional closure part;

FIG. 8 a central longitudinal section of the device of FIG. 7;

FIG. 9 a lateral view of the device of FIG. 6 with a hollow needle connected;

FIG. 10 a lateral view of the device of FIG. 6 with a mixing tube connected;

FIG. 11 a central longitudinal section of the device of FIG. 10; and

FIG. 12 a central longitudinal section of a device with a housing, piston, and a mixing tube integrally designed with a closure cap.

### DESCRIPTION OF PREFERRED EMBODIMENTS

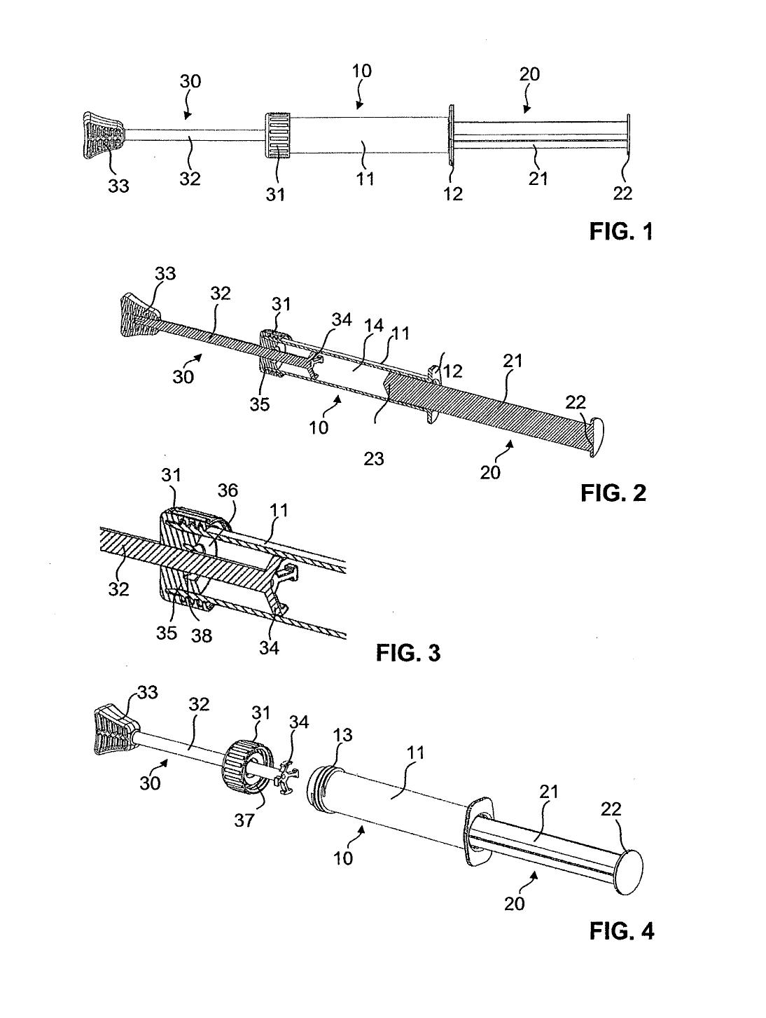

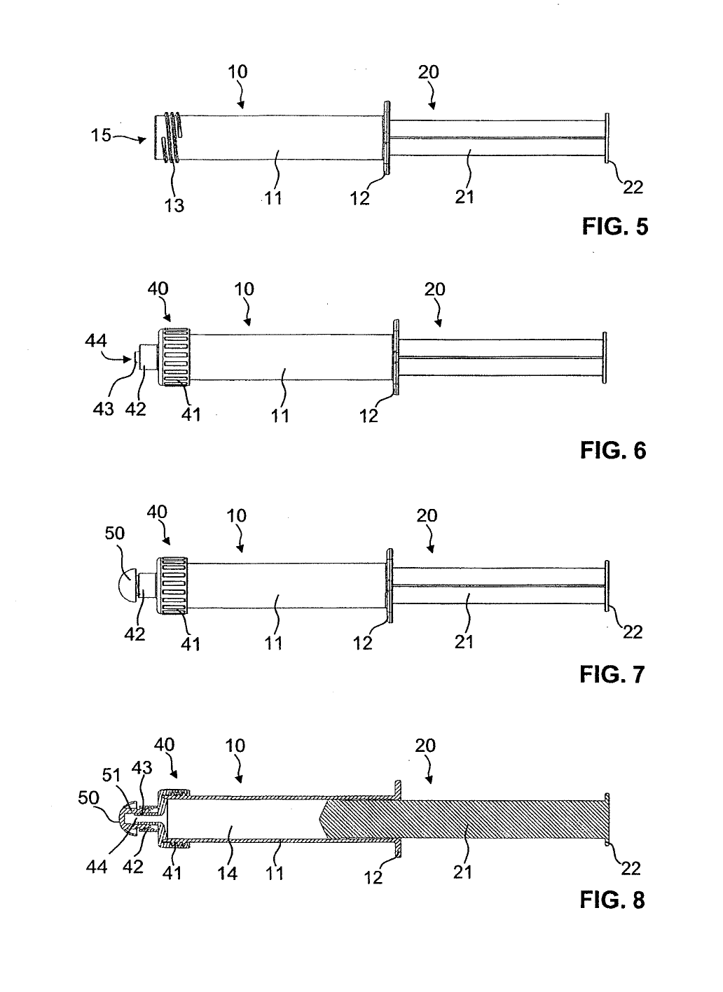

FIGS. 1 to 4 illustrate a preferred exemplary embodiment of a mixing device according to the invention. The mixing device comprises a housing 10 whose sidewall 11 delimits a cylindrical mixing chamber 14. The mixing chamber 14 defines a longitudinal direction by its central axis, and comprises a distal open end 15 (FIG. 5) and a proximal open end.

A piston unit 20 has been inserted into the mixing chamber 14 through the proximal open end. The piston unit comprises a piston 23 which rests against the sidewall 11 (i.e. the defining wall of the mixing chamber 14) in a circumferentially sealing manner, a piston rod 21 connected to the aforesaid with a cruciform cross section, as well as a thumb rest 22 formed on the proximal end of the piston rod 21. FIG. 5 shows the housing 10 and the piston unit 20 on their own.

The open distal end 15 of the mixing chamber 14 is closed by a first closure cap 31 that is screwed onto an external thread 13. The closure cap 31 comprises a sleeve-like or bush-like guide element 35 which axially extends in the proximal direction into the mixing chamber 14 and with its outer circumferential surface rests against the interior of the sidewall 11 of the housing 10. The guide element 35 has a central hole along the longitudinal direction, in which hole an operating rod 32 is guided.

At the proximal end of the operating rod 32 a mixing element 34 is formed in a single part with the operating rod. The mixing element 34 comprises four arms that from the centre axis extend radially outwards, which arms are arranged at an angle of approx. 60 to 75 degrees to the longitudinal direction, and are inclined towards the proximal direction. At their free ends the arms comprise a T-shaped thickening. With these free ends, on the inside, the arms rest against the sidewall 11 of the housing 10.

A proximal end region of the guide element 35 comprises a flat, conical indentation 36 which is distally joined by a cylindrical indentation, in the manner of a blind hole, of smaller diameter. The cylindrical indentation in turn comprises a circumferential wiping lip 38. The wiping lip 38 rests against the operating rod so as to form a seal. Within the cylindrical indentation, starting from the distal face of the indentation, said wiping lip 38 essentially protrudes in the proximal direction, wherein the thickness of the wiping lip tapers off in the proximal direction.

The shape of the flat indentation 36 is complementary to the shape of the mixing element 34 in such a manner that the mixing element 34 is partly received in the indentation 36 when said mixing element 34 is entirely withdrawn to a distal end position by means of the operating rod 32. Conversely, the distal end region of the piston 23 is also designed so as to be complementary to the shape of the mixing element 34 in such a manner that the arms of the mixing element 34 are supported by the distal end region of the piston 23 when the mixing element 34 is pushed by means of the operating rod into a proximal end position in which it rests against the piston 23. In other words, in this position the mixing element forms a receiving device for that end of the piston 23 which extends in the distal direction.

A hand grip 33 has been affixed, in particularly clipped on or pressed on, to the distal end of the operating rod 32. The hand grip 33 comprises a flattened shape and opens out in a wedge-shaped manner towards the distal side. Consequently, at its distal end it forms a relatively large supporting surface for the ball of the thumb of one hand of the user.

The mixing element 34, the operating rod 32, the hand grip 33 and the first closure cap 31 together form a mixing unit 30. The latter is installed during manufacture in that at first the operating rod 32 with the single-piece mixing element 34 affixed thereto is inserted from the proximal side through the passage opening of the guide element 35, and subsequently the hand grip 33 is clicked onto the distal end of the operating rod 32. The entire mixing unit is removable as an entity from the housing 10, as is indicated in FIG. 4.

In order to produce a mixture of several components with this mixing device, at first the components are placed into the mixing chamber 14 with the piston unit 20 withdrawn and the mixing unit 30 removed. Subsequently, the mixing unit 30 is placed onto the open end 15 of the mixing chamber 14, and the first closure cap 31 is screwed onto the thread 14. In order to mix the components, the mixing element 34, in the mixing chamber 14, is then moved to and fro along the longitudinal direction and is rotated about the longitudinal direction. To this effect the user holds the housing 10 with their first hand while operating the hand grip 33 with their second hand. When the product to be mixed has been adequately mixed, the user withdraws the operating rod 32 in the distal direction until the mixing element 34 comes to rest against the guide element 35 of the closure cap 31. Subsequently, the user unscrews the closure cap 31 from the housing 10 and removes the complete mixing unit 30.

In order to then discharge the product from the mixing chamber 14 the user places a second closure cap 40 onto the distal open end of the mixing chamber 14. This is illustrated in FIG. 6. The second closure cap 40, like the first closure cap 31, comprises a jacket region 41 on which an internal thread is formed that engages the external thread 13 on the housing 10. The closure cap 40 comprises a discharge opening 44 that extends in longitudinal direction along the central axis of the mixing chamber 14 and that is encompassed by a connecting region 43 in the form of a male Luer cone. A securing sleeve 42 that is firmly connected to the closure cap 40 radially encompasses the connecting region 43. On the inside of the securing sleeve 42 an internal thread is formed. The connecting region 43 and the securing sleeve 42 together form a male Luer-lock connection for an accessory part.

If the discharge opening 44 is to be closed, to this effect a closure part 50 can be used, as illustrated in FIGS. 7 and 8. The closure part 50 comprises a sleeve 51 whose inner circumferential surface is designed so as to be complementary to the Luer cone of the connecting region 43.

On its outer circumferential surface the sleeve 51 comprises an external thread that can be screwed into the internal thread of the securing sleeve 42. In the distal direction the sleeve 51 is closed by a mushroom-shaped or hemispherical terminal piece.

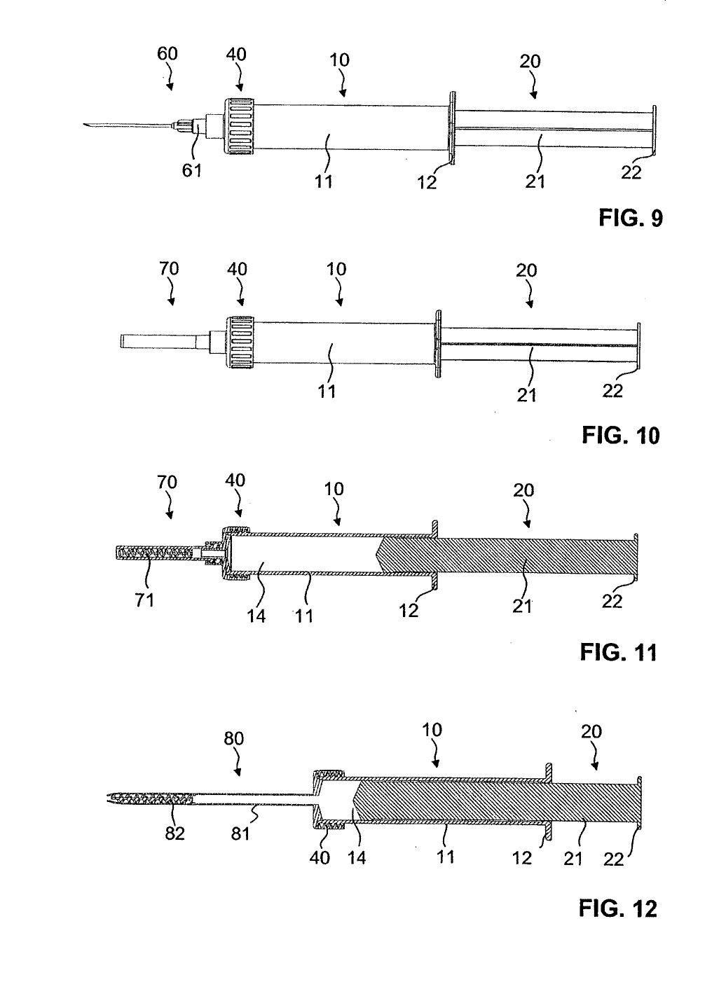

In order to be able to discharge the product from the mixing chamber 14, various accessory parts can be slipped onto the connecting region 43 and secured in the connecting region by means of the securing sleeve 42. This is illustrated in FIGS. 8-12. The accessory part can, for example, be a commercially available hollow needle 60 (FIG. 9) with a connecting region 61 that is complementary to the connecting region 43 of the second closure cap. As an alternative, the accessory part can be a mixing tube 70 (FIGS. 10 and 11). The mixing tube 70 can, in particular, comprise a further mixing element 71, for example in the form of a static mixing spiral, in order to achieve final mixing of the product to be mixed, while said product is being discharged. A multitude of other accessory parts are imaginable.

The mixing device, together with the second closure cap, with one or several such accessory parts, and if applicable with the closure part, forms a complete mixing and discharge system that can be marketed as a complete set.

The accessory part can also be designed in a single piece with the second closure cap 40, as illustrated in FIG. 12 in the context of an accessory part 80. In this embodiment a mixing tube 81 is connected in one piece with the second closure cap 40, in which mixing tube 81 in a distal end region a mixing spiral 82 is arranged. In this embodiment, too, a multitude of further accessory parts are imaginable.

In order to discharge from the mixing chamber 14 the mixed product, the user holds the housing 10 between two fingers, in particular the index finger and the middle finger, of one hand. With the thumb of the same hand the user exerts force, which acts in the distal direction, on the thumb rest 22. To make it possible to better absorb the experienced counter force with the fingers without sliding, a retaining flange 12, known per se, is provided on the proximal end of the housing 10. Discharging the mixture from the mixing device thus essentially takes place in the same manner as administering a substance from a commercially available injection syringe.

The mixing device presented above and the mixing and discharge system based thereon are particularly suited for the production of relatively small quantities of a mixed product. In particular, the mixing chamber 14 preferably has a volume of between approx. 1 millilitre and 100 millilitres. Thus the mixing device is particularly suitable for use in the medical field, in particular for producing small quantities of bone cement, dental mass, medication that is to be administered orally, etc. Because of its simple construction and low cost of production, the system is well-suited to be used as a disposable product for single use.

Of course, a multitude of modifications of the mixing device and of the mixing and discharge system according to the invention are possible. For example, it is also possible to produce the mixing element separately from the operating rod, in particular also from some other material. Of course, other shapes of the housing 10, of the piston unit 20 and of the mixing unit 30, in particular the hand grip 33, are possible. The connection between the closure caps and the housing can be implemented in a manner that differs from that shown in this document, for example by way of a bayonet-like connection. A multitude of further modifications are possible, and the invention is by no means restricted to the examples provided above.

### LIST OF REFERENCE CHARACTERS

10 Housing
11 Sidewall
12 Retaining flange
13 External thread
14 Mixing chamber
20 Piston unit
21 Piston rod
22 Thumb rest
23 Piston
30 Mixing unit
31 First closure cap
32 Operating rod
33 Hand grip
34 Mixing element
35 Guide element
36 Indentation
37 Internal thread
38 Wiping lip
40 Second closure cap
41 Jacket region
42 Securing sleeve
43 Connecting region
44 Discharge opening
50 Closure part
51 Sleeve
60 Hollow needle
61 Connecting region
70 Mixing tube
71 Mixing spiral
80 Accessory part
81 Mixing tube
82 Mixing spiral

1-15. (canceled)

16. A mixing and discharge system for mixing and discharging a product to be mixed, comprising:
a housing that delimits an essentially cylindrical mixing chamber, wherein the mixing chamber defines a longitudinal direction and comprises a distal open end and a proximal open end, and wherein the housing is dimensioned in such a manner that it can be held between two fingers of a first hand of a user;
a piston arranged in the mixing chamber so as to be slidable along the longitudinal direction, which piston sealingly delimits the mixing chamber in the direction of the proximal end;
a piston rod, connected to the piston, which piston rod protrudes from the proximal end of the mixing chamber and which piston rod comprises a thumb rest, wherein the thumb rest is arranged in such a manner that it makes it possible for the user to advance the piston rod with the thumb of the first hand in the distal direction when the user holds the housing between two fingers of the first hand;
a mixing element that is configured to be slid in the mixing chamber along the longitudinal direction; and
an operating rod that is distally connected to the mixing element, which operating rod protrudes from the distal open end of the mixing chamber;
a first closure cap that at the distal open end of the mixing chamber detachably engages the housing, which closure cap closes the distal open end of the mixing chamber and comprises a passage opening through which the operating rod is guided, the first closure cap together with the operating rod and the mixing element being completely removable from the housing (10); and
a second closure cap (40), which, after removal of the first closure cap (31), in the region of the distal open end (15) of the mixing chamber (14) is configured to detachably engage the housing (10) in order to close the distal open end (15) of the mixing chamber (14), and which comprises a discharge opening (44) through which a mixed product present in the mixing chamber (14) can be discharged in the distal direction with the application of pressure on the piston rod (21).

17. The mixing and discharge system according to claim 16, wherein the first closure cap comprises a guide element that extends into the mixing chamber, which guide element rests against at least three positions, preferably circumferentially, on the inside against a defining wall of the mixing chamber, with the operating rod being guided in said guide element.

18. The mixing and discharge system according to claim 16, wherein a proximal end region of the first closure cap comprises an indentation that tapers off in the distal direction in order to receive a distal end of the mixing element when the mixing element is in a distal end position.

19. The mixing and discharge system according to claim 16, wherein the piston comprises an end region that tapers off in the distal direction, and wherein the mixing element forms a distally-tapering receiving device for the end region of the piston.

20. The mixing and discharge system according to claim 16, wherein the first closure cap comprises a circumferential wiping lip that rests against the operating rod.

21. The mixing and discharge system according to claim 16, wherein the mixing device comprises a hand grip that is connected to the operating rod at its distal end that faces away from the mixing element, and wherein the hand grip is designed, by way of the second hand of the user, to manually move the operating rod to and fro along the longitudinal direction and to rotate it while the user holds the housing with the first hand.

22. The mixing and discharge system according to claim 21, wherein the hand grip is designed so as to open out in a wedge-shaped manner in the distal direction and so as to be asymmetrical relative to a rotation on the longitudinal direction.

23. The mixing and discharge system according to claim 16, wherein the operating rod is non-detachably connected to the mixing element.

24. The mixing and discharge system according to claim 16, wherein an external thread is formed on the housing in the region of the distal end of the mixing chamber, and wherein the first closure cap comprises a jacket region with an internal thread that engages the external thread.

25. The mixing and discharge system according to claim 16, wherein the open distal end (15) of the mixing chamber (14) forms an access opening to the mixing chamber (14), with the diameter of said access opening being at least as large as the diameter of the mixing chamber (14) in a cylindrical region that is situated so as to be proximally spaced apart from the distal end (15), in which cylindrical region the piston (23) is slidable.

26. The mixing and discharge system according to claim 16, wherein the second closure cap comprises a connecting region for an accessory part, which connecting region encompasses the discharge opening, and comprises a jacket surface that conically extends in the distal direction and that is preferably designed as a male Luer cone.

27. The mixing and discharge system according to claim 26, wherein the second closure cap comprises a securing sleeve which radially encompasses the connecting region and which is arranged so as to be non-rotational or rotational, with an internal thread facing the connecting region in order to secure the accessory part on the connecting region.

28. The mixing and discharge system according to claim 26 which, furthermore, comprises a closure part that is configured to be detachably attached to the connecting region in order to close the discharge opening.

29. The mixing and discharge system according to claim 16 which, furthermore, comprises at least one accessory part that is configured to be detachably connected to the discharge opening or that is non-detachably connected to the discharge opening, and that is selected from the following group of accessory parts:
a discharge nozzle;
a spraying device;
a hollow needle;
a mixing tube.
